(19) United States
(12) Patent Application Publication
(10) Pub. No.: US 2012/0052565 A1
(43) Pub. Date: Mar. 1, 2012

(54) Porous and Non-Porous Cell Culture Substrate

(76) Inventors: Michael Edward DeRosa, Painted Post, NY (US); Joydeep Lahiri, Painted Post, NY (US); Hongwei Hanna Rao, Horseheads, NY (US); Po Ki Yuen, Painted Post, NY (US)

(21) Appl. No.: 13/218,103

(22) Filed: Aug. 25, 2011

Related U.S. Application Data
(60) Provisional application No. 61/377,633, filed on Aug. 27, 2010.

(51) Int. Cl. C12M 3/00; C23F 1/00

(52) U.S. Cl. 435/305.1; 216/24

(57) ABSTRACT

A cell culture article has substrate that is predominantly opaque and that provides a three-dimensional (e.g., irregular) surface, but incorporates an optically transparent, substantially regular (e.g., two-dimensional) surface to serve as a microscopic observation and imaging window for the 3D cell culture. In many embodiments, the 3D portion of the substrate occupies greater than 99% of the surface while the 2D portion occupies less than 1% of the surface so as not to substantially disrupt the general 3D culture environment.

### CROSS-REFERENCE TO RELATED APPLICATIONS

This application claims the benefit of priority under 35 U.S.C. §119 of U.S. Provisional Application Ser. No. 61/377,633 filed on Aug. 27, 2010 the content of which is relied upon and incorporated herein by reference in its entirety.

### FIELD

The present disclosure relates to cell culture articles with three-dimensional substrates and methods of making the same.

### BACKGROUND

Most adherent cell cultures have been developed on two-dimensional (2D) flat surfaces. Deciphering the mechanisms of cell behavior and controlling cell fate for therapeutic applications in vitro often requires three-dimensional (3D) cell culture systems to provide physiologically relevant microenvironments. However, many 3D cell culture surfaces are not optically transparent due to light scattering from the substrate. It can be difficult to perform routine microscopic observation of cells cultured on these surfaces using conventional inverted or upright light microscopes. The direct light microscopic observation of cells in cell culture is important for obtaining the dynamic information of cell morphology, physiology and behavior. In addition, monitoring the cells with light microscopy at each handling of a culture is important to identify visible microbial contamination.

Confocal microscopy and optical coherence tomography provide some ability to examine cells cultured on 3D substrates. However, these imaging techniques can be limited due to the dense and opaque nature of such substrates. An electron microscope has significantly greater magnification and resolution than a light microscope and some types can produce spectacular 3D images, but it is not suitable for routine living cell culture observation as it requires that the specimen be fixed. An atomic force microscope (AFM) can be used for living cell culture without the fixation of cells when it is set to a non-contact mode of operation, but the cell culture samples need to be opened on the observation stage since the AFM detection needs to have the cantilever close to the samples. Opening cell cultures outside of a laminar flow hood usually is undesirable for cell culture aseptic technique. Thus, AFM is generally not a usable technique for the routine imaging and observation of 3D cell culture either.

### BRIEF SUMMARY

The present disclosure describes, among other things, a cell culture article having substrate that is predominantly opaque and that provides a 3D (e.g., irregular) surface, but incorporates an optically transparent, substantially regular (e.g., two dimensional) surface to serve as a microscopic observation and imaging window for the 3D cell culture. In many embodiments, the 3D portion of the substrate occupies greater than 99% of the surface while the 2D portion occupies less than 1% of the surface so as not to substantially disrupt the general 3D culture environment.

In various embodiments described herein, a cell culture article includes a three dimensional substrate having a surface for culturing cells. The substrate has one or more optically opaque regions and one or more optically transparent regions. The optically opaque regions occupy 75 percent or more of the surface of the substrate, and at least one of the optically transparent regions occupies an area of the surface sufficient for viewing cells via a light microscope.

In various embodiments described herein, a method for fabricating a cell culture substrate having one or more optically opaque regions and one or more optically transparent regions is described. The method includes (i) applying mask to an optically transparent substrate to produce a masked substrate having a masked and an unmasked portion; (ii) applying a treatment to the masked substrate to render the unmasked portion optically opaque; and (iii) removing the mask.

The devices, articles and methods described herein may provide one or more advantages over prior cell culture articles having three-dimensional substrates. For example, embodiments of the cell culture articles and substrates described herein have a window for microscopic observation and imaging of 3D cell culture by incorporating an optically transparent surface into opaque 3D substrates, allowing for inexpensive and practical methods to obtain basic information regarding cell morphology, physiology and behavior using light microscopes rather than more expensive specialized microscopes. The “windows” also allow for routine examination for microbial contamination, which can allow for prevention of contamination spread, saving time and expense. In many embodiments, the three dimensional surface can be added to existing articles, avoiding the need to substantially change manufacturing processes to fabricate the 3D substrates. These and other advantages of the various embodiments of the devices and methods described herein will be readily apparent to those of skill in the art upon reading the disclosure presented herein.

### BRIEF DESCRIPTION OF THE DRAWINGS

FIG. 1 is a schematic top view of a cell culture substrate having a predominantly 3D surface and an optically transparent 2D region.

FIG. 2 is a schematic top view of a plate having six substrate regions for use in a 6-well cell culture article.

FIG. 3 is a schematic, diagrammatic depiction of a method for generating a cell culture substrate as depicted in FIG. 1.

FIGS. 4A-C depicts photographic images of 6-well cell culture plates at various stages of a process as depicted in FIG. 3.

FIG. 5A is a photographic image of a 6-well culture plate having a surface generated according to an embodiment of a method described herein.

FIG. 5B is a scanning electron micrograph of the depicted portion of the cell culture surface of FIG. 5A.

FIGS. 6A-D are light microscope images of MC3T3 cells cultured 2 hours (A and B) and 2 days (C and D) after seeding the cells in growth medium on an embodiment of a cell culture article having an optically transparent portion. The width of the optically transparent portion in FIGS. 6A and 6C are 2 mm. In FIGS. 6B and 6D, the width is 0.3 mm.

FIG. 7 is a bar graph showing the results of a LDH assay of MC3T3 cells after 5 days of cell culture on cell culture article having various substrates.

The schematic drawings presented herein are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to.” It will be understood that the terms “consisting of” and “consisting essentially of” are subsumed in the term “comprising,” and the like. For example, a three dimensional cell culture substrate comprising one or more optically opaque regions and one or more optically transparent regions may consist of, or consist essentially of, optically opaque regions and the optically transparent regions.

“Consisting essentially of”, as it relates to a compositions, articles, systems, apparatuses or methods, means that the compositions, articles, systems, apparatuses or methods include only the recited components or steps of the compositions, articles, systems, apparatuses or methods and, optionally, other components or steps that do not materially affect the basic and novel properties of the compositions, articles, systems, apparatuses or methods.

Any direction referred to herein, such as “top,” “bottom,” “left,” “right,” “upper,” “lower,” “above,” below,” and other directions and orientations are described herein for clarity in reference to the figures and are not to be limiting of an actual device or system or use of the device or system. Many of the devices, articles or systems described herein may be used in a number of directions and orientations.

As used herein, “microporous structure” refers to a structure having pores or interstices of an average diametric size of less than 1000 micrometers.

As used herein, “pore” means a cavity or void in a surface, a body, or both a surface and a body of a solid article, where the cavity or void has at least one outer opening at a surface of the article.

As used herein, “interstice” means a cavity or void in a body of a solid polymer not having a direct outer opening at a surface of the article, i.e., not a pore, but may have an indirect outer opening or pathway to an outer surface of the article by way of one or more links or connections to adjacent or neighbor “pores” “interstices,” or a combination thereof.

As used herein, a “solvent” for a polymeric sheet is a composition capable of causing swelling or solubilization of at least a portion of the polymeric sheet when contacted with the sheet. A “non-solvent” for a polymeric sheet means a composition that does not cause swelling or solubilization of the polymeric sheet when contacted with the sheet.

As used herein, an object that is “optically transparent” means an object through which light transmits without appreciable scattering so that a body laying beyond the object is capable of being seen clearly. For the purposes of cell culture, an optically transparent article is an article on which cultured cells can be suitably observed via a standard light microscope. While not intending to be bound by theory, three dimensional or irregular surfaces may scatter light such that images of cells viewed by standard light microscopy techniques are distorted to an extent that cell morphology or cellular structures cannot be readily identified or such that images of cells cannot be seen at all. In many cases, a cell culture substrate that transmits 50% or more of visible light through substrate is optically transparent.

As used herein, an object that is “optically opaque” is an object scatters light so that a body laying beyond the object cannot be seen clearly seen. For cell culture articles, an opaque substrate is a substrate on which the morphology of cultured cells cannot be suitably observed via a standard light microscope.

The present disclosure describes, among other things, cell culture articles having substrates that are predominantly opaque and which provide a three-dimensional surface. The substrates incorporate optically transparent, substantially regular regions (e.g., surfaces to serve as a microscopic observation and imaging window for the three-dimensional cell culture). In many embodiments, the three-dimensional portion of the substrate occupies greater than 99% of the surface while the two-dimensional portion occupies less than 1% of the surface so as not to substantially disrupt the general three-dimensional culture environment. Accordingly, chemical and biological assays may be readily performed to determine the performance of the cells grown on the three dimensional surface. In addition, it is still possible to use more sophisticated microscopy techniques to image cells on the three-dimensional portions structures, if desired.

### 1. Cell Culture Substrate

The substrates described herein may be employed with or incorporated into any suitable cell culture article. For example, Petri dishes, multi-well plate such as 6, 24, and 96-well plates, cell culture flasks, or other cell culture articles may include a cell culture substrate as described herein.

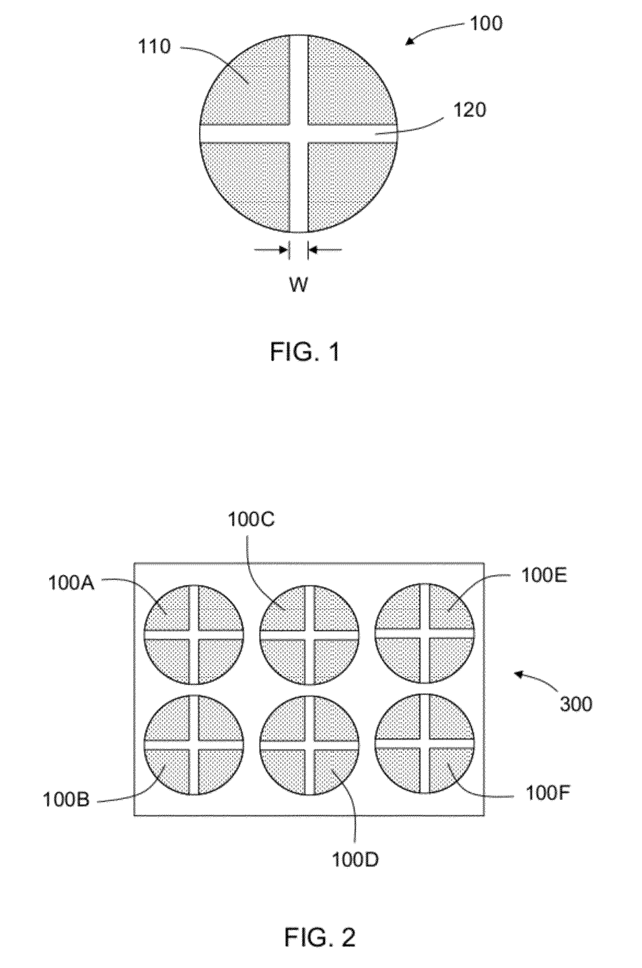

Referring now to FIG. 1, a schematic perspective view of a cell culture substrate 100 is shown. The substrate 100 has one or more optically opaque regions 110 and one or more optically transparent regions 120. The opaque regions 110 have an irregular (three-dimensional) surface to provide a more physically relevant cell culture substrate. The majority of the surface of the substrate 100 is occupied by the opaque region 110. Accordingly, the overall substrate 100 is considered herein to be a three dimensional substrate. In various embodiments, the opaque regions 110 occupy 75% or more of the surface of the substrate; e.g., 90% or more, 95% or more, or 99% or more of the surface of the substrate. For the purposes of this disclosure, the percentage of the surface area of the substrate 100 that the opaque regions 110 or the transparent regions 120 occupy are determined by assuming the surface is flat and regular, even though the surface of the opaque regions 110 are irregular and three dimensional. That is, the irregularity of the surface area of the opaque 110, and possibly the transparent 120, regions is not accounted for in determining the percent of the surface of the substrate 100 occupied by the opaque regions 110 or the transparent regions 120.

At least one, if not all or substantially all, of the one or more transparent regions 120 occupy an area of the surface of the substrate 100 to allow viewing of cells cultured on the transparent region to be viewed via a light microscope. Preferably, the cells are viewable in a transparent region 120 under a variety of magnifications typically used for cell culture, such as between 5× and 50×. For example, the transparent regions may occupy an area of 0.025 mm² or greater, such as 0.04 mm² or greater or 0.09 mm² or greater, of the surface area of the substrate 100. In the embodiment depicted in FIG. 1, the optically transparent region 120 has a length and a width W. It has been found that widths W as small as 0.3 mm are sufficient for viewing cells with light microscopy. However, it is believed that smaller width may also be suitable, such as widths of about 0.2 mm. Of course, the substrate 100 may have one or more optically transparent regions 110 having any suitable length and width.

The transparent regions 120 in FIG. 1 form two rectilinear perpendicular lines extending the length of the substrate 100 in a cross or plus shaped manner. One advantage of having a transparent region 120 that extend the length of the substrate 100 is that it allows for viewing of cells in the middle as well as at the edges of the substrate 100. Of course, this can also be accomplished by multiple smaller transparent regions with one or more disposed at or near the middle, and one or more disposed at or near the edge.

It will be understood that the transparent region 120 pattern and shape depicted in FIG. 1 is one example of the many, if not infinite, patterns and shapes that the transparent regions 120 may occupy with regard to the substrate 100. For the example, the substrate 100 may include one or more circular, rectangular, square, oval, triangular or otherwise shaped transparent regions 120. Preferably, at least one, if not all or substantially all, of the transparent regions 120 provide a window for light microscopic viewing or imaging of cells cultured on the substrate 100 at a variety of magnifications.

The substrates described herein may be employed with or incorporated into any suitable cell culture article. For example, a substrate having one or more optically opaque three dimensional regions and one or more optically transparent regions can be formed in an existing cell culture article, can be formed separately and assembled into a cell culture article, or can serve as a cell culture article itself.

An example of an article having multiple substrates for cell culture is shown in FIG. 2. The article 300 is a base plate for a 6 well cell culture plate. The article 300 includes six substrates 100A-F for cell culture that will correspond to the bottoms of the wells of the 6 well culture plate when fully assembled. Each substrate 100A-F has one or more optically opaque regions and one or more optically transparent regions. Thus, “substrate” as used herein is limited to the region on which cells are intended to be cultured.

### 2. Formation of Three Dimensional Substrate

Any suitable method may be used to form a three dimensional substrate having optically transparent regions to allow for microscopic examination or imaging of cells cultured on the substrate.

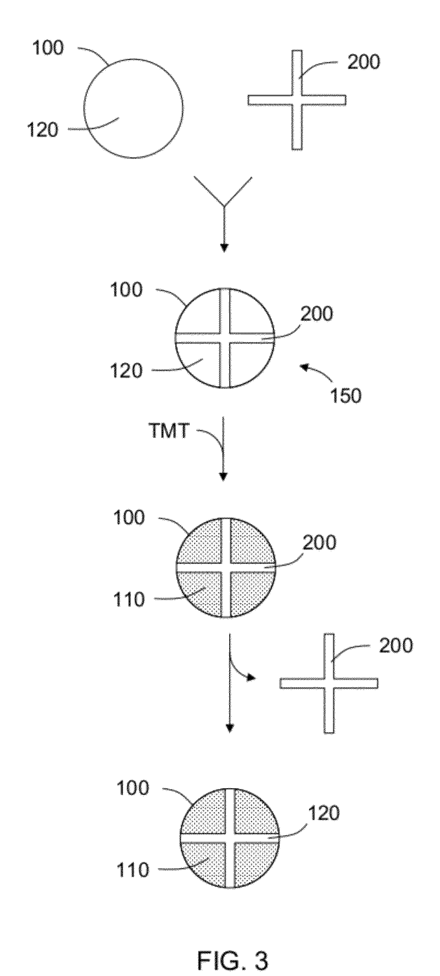

One suitable method is diagrammatically depicted in FIG. 3. In FIG. 3, a mask 200 is applied to an optically transparent 120 article 100 to produce a masked article 150 having masked and unmasked portions. The surface of the masked article 150 may then be subjected to a treatment (TMT) to render the unmasked portions of the article 100 three dimensional, and thus optically opaque 120. The mask 200 may be removed, yielding a substrate 100 having three-dimensional opaque regions 110 and transparent regions 120.

Any suitable mask may be used. The mask 200 should prevent the surface of the article 100 from being rendered opaque by the treatment (TMT). Additionally, the mask 200 should be readily removable from the article 100 following the treatment (TMT). In many embodiments, a stencil, self adhesive tape or other film is used as a mask.

One convenient way to form a film mask with a desired pattern is to use a desktop digital cutting device, such as described in, for example, P. K. Yuen and V. N. Goral, “Low-cost rapid prototyping of flexible microfluidic devices using a desktop digital craft cutter”, Lab on a Chip, 2010, 10, 384-387. Of course, any other suitable method may be used to cut or produce a mask to a desired pattern.

Any suitable treatment (TMT) may be applied to the masked article 150 to render the unmasked portions three dimensional, and thus opaque. For example, a coating may be applied using an appropriate technique to render the unmasked portions three dimensional, electrospun randomly oriented nanofibers of, for example, polyamide may be applied to the unasked portions, glass fibers; e.g., porous glass fibers, may be applied to the unmasked surface, or the like.

In various embodiments, the unmasked portions of the surface are rendered microporous. Any suitable methods for rendering the surface microporous may be used. For example, a solvent based process as described in co-pending U.S. patent application Ser. No. 13/218,002, entitled MICROPOROUS THERMOPLASTIC ARTICLE, filed on the same date as the present application, naming Michael E. DeRosa and Mircea Despa as inventors, and having attorney docket number SP10-243, which application is hereby incorporated by reference in its entirety to the extent that it does not conflict with the present disclosure, may be employed. In this method, a thermoplastic article having a birefringence of 0.0001 or greater, such as 0.001 or greater or 0.01 or greater, is contacted with a composition having a solvent for the thermoplastic polymer, and the composition is removed, yielding a microporous surface on the article. The composition has a solvent strength sufficient to swell the polymer but not to dissolve the polymer. A non-solvent may be added to the composition to achieve an appropriate ratio of solvent and non-solvent to produce the desired microporous surface.

The composition comprising the solvent may include one or more solvents and one or more non-solvents. As generally understood in the art, different polymeric materials are soluble or swellable in different solvents. Accordingly, the one or more solvents employed will be dependent on the polymeric material of the article. Any solvent suitable for solubilizing or swelling a polymer of the article may be employed. Such solvents are generally known in the art. For example, for polystyrene, suitable solvents include tetrahydrofuran, methylethyl ketone, acetone, and ethyl acetate. For cyclic polyolefins suitable solvents include methylene chloride, and tetrahydrofuran. For styrene maleic anhydride polymeric sheets, suitable solvents include acetone, tetrahydrofuran, 1,3-dioxolane, methylethyl ketone, toluene, ethyl acetate, N-methylpyrolidone. It will be understood that these are only a few examples of the suitable solvents that may be used for these polymers and that other solvents may readily be used and that other polymers with appropriate solvents may be used in accordance with the teachings herein to generate a microporous structure.

By way of example, it has been found that solvent compositions having the following ratios, on a volume/volume basis, of solvent and non-solvent are suitable for forming microporous structures from polystyrene articles having a birefringence of 0.0001 or greater:tetrahydrofuran (THF)/isopropanol in range of 35/65-50/50; THF/ethanol in a range of 35/65-50/50; ethyl acetate/isopropanol in a range of 45/55-65/35; and THF/water in a range of 45/55-65/35. By way of further example, it has been found that solvent compositions having the following ratios, on a volume/volume basis, of solvent and non-solvent are suitable for forming microporous structures from cyclic olefin copolymer articles having a birefringence of 0.0001 or greater:methylene chloride (single solvent); THF/isopropanol in a range of 75/25-90/10; and THF/water in a range of 90/10-98/2. It will be understood that these are just examples that were found to work successfully and these do not constitute an exhaustive list of solvents, non-solvents, and polymers for which the processes described herein will produce microporous surface structures.

The solubility strength required to induce micropore formation will depend on the nature of the polymer employed and the residual stress in the polymeric article, with a lower solvent strength needed when the article has higher residual stress or birefringence. A solvent strength of a solvent or solvent mixture that is suitable for inducing pore formation on a polymeric article may be determined using Hansen solubility parameters (see, e.g., Hansen, C. M., Hansen Solubility Parameters a User's Handbook 2nd Ed., CRC Press, Boca Raton, 2007). We have found that solvent or solvent mixtures that have Hansen Relative Energy Difference (RED) values in a range of the polymer solubility boundary have been found to cause microporous formation on molded thermoplastic articles. In particular, fluid compositions comprising one or more solvents, which may also contain one or more non-solvents, that have a RED of between about 0.5 and about 2 may be suitable for forming microporous structures on polymeric articles. Preferably, the fluid composition has a RED of between about 0.75 and about 1.6, such as between about 0.8 and about 1.5 or between about 0.85 and about 1.45.

A more detailed discussion of Hansen solubility parameters and RED is discussed in co-pending U.S. patent application Ser. No. 13/217,818, entitled MICROPOROUS THERMOPLASTIC SHEETS, having attorney docket no. SP11-197, naming Michael DeRosa, Todd Upton, and Ying Zhang as inventors, and filed on the same date herewith, which application is hereby incorporated herein by reference in its entirety to the extent that it does not conflict with the disclosure presented herein.

According to Hansen, the total cohesion energy (E) of a liquid is defined by the energy required to convert a liquid to a gas. This can be experimentally measured by the heat of vaporization. Hansen described the total cohesion energy as being comprised of three primary intermolecular forces: atomic dispersion forces (E_{D}), molecular permanent dipole-dipole interactions (E_{P}), and molecular hydrogen bonding interactions (E_{H}). When the cohesion energy is divided by the molar volume (V) the total cohesive energy density of the liquid is given by

E/V=E_{D}/V+E_{P}/V+E_{H}/V. (1)

The solubility parameter (δ) of the liquid is related to the cohesive energy density by

δ=(E/V)^{1/2} (2)

where δ is the Hildebrand solubility parameter. The three Hansen solubility components of a liquid are thus given by

δ²=δ_{D}²+δ_{P}²+δ_{H}². (3)

These three parameters have been tabulated for thousands of solvents and can be used to describe polymer-solvent interactions (see, e.g., Hansen, 2007).

Solubility parameters exist for solid polymers as well as liquid solvents (see, e.g., Hansen, 2007). Polymer-solvent interactions are determined by comparing the Hansen solubility parameters of the polymer to that of a solvent or solvent mixture defined by the term Rₐ as

R^{a}=4(δ_{D2}−δ_{D1})²+(δ_{P2}−δ_{P1})²+(δ_{H2}−δ_{H1})² (4)

where subscripts 1 and 2 refer to the solvent or solvent mixture and polymer respectively. Rₐ is the distance in three dimensional space between the Hansen solubility parameters of a polymer and that of a solvent. A “good” solvent for a particular polymer has a small value of Rₐ. This means the solubility parameters of the polymer and solvent are closely matched and the solvent will quickly dissolve the polymer. Rₐ will increase as a solvent's Hansen solubility parameters become more dissimilar to that of the polymer.

The solubility of a particular polymer is not technically described by just the three parameters in Equation (3). A good solvent does not have to have parameters that perfectly match that of the polymer. There is a range of solvents that will work to dissolve the polymer. The Hansen solubility parameters of a polymer are defined by δ_{D}, δ_{P}, and δ_{H} which are the coordinates of the center of a solubility sphere which has a radius (Rₒ). Rₒ defines the maximum distance from the center of the sphere that a solvent can be and still dissolve the polymer.

The strength of a solvent for a polymer is determined by comparing Rₐ to Rₒ. A term called the Relative Energy Difference (RED) is given by

RED=Rₐ/Rₒ. (5)

Using RED values is a simple way to evaluate how “good” a solvent will be for a given polymer. Solvents or solvent mixtures that have a RED number much less than 1 will have Hansen solubility parameters close to that of the polymer and will dissolve the polymer quickly and easily. Liquids that have RED numbers much greater than 1 will have Hansen solubility parameters further away from the polymer and will have little or no effect on the polymer. Liquids that have RED numbers close to one will be on the boundary between good and poor solvents. These liquids usually swell the polymer and belong to a class of solvents that typically cause environmental stress cracking and crazing (see, e.g., Hansen, C. M.; Just, L., “Prediction of Environmental Stress Cracking in Plastics with Hansen Solubility Parameters, Ind. Eng. Chem. Res., 40, 21-25, 2001).

It will be understood that the width of suitable RED value ranges for inducing pore formation depend on the amount of residual stress in the polymer article, with higher residual stress resulting in higher RED values. That is, the higher the amount of residual stress, or birefringence, the larger the RED value will be for the upper boundary. Polymeric articles that have lower stress or birefringence will require solvents or solvent mixtures that are closer to the center of the sphere within the shaded region to produce porous surfaces.

It will also be understood that the values of R₀ value of a given polymer may change depending on the amount of residual stress or birefringence of the article. The value obtained for R₀ may also change based on the solvents or non-solvents used to determine the R₀ value. If solvents or combinations of solvents and non-solvents are used that are within the micropore forming range (e.g. shaded area of the sphere in FIG. 2), then the value of R₀ may more readily change depending on residual stress or birefringence. However, if solvents or combinations of solvents and non-solvents are used that are not within the micropore forming range, the determined R₀ value may not change with changing residual stress or birefringence values. The non-porous starting thermoplastic article may be contacted with the composition comprising solvent and non-solvent in any suitable manner. For example, the article may be submersed into the liquid composition, the composition may be sprayed on, dropped on, pipetted on, cast on, poured on, or otherwise applied to the article, the composition maybe vaporized and applied to the article, and the like. It has been found that dipping the article into the liquid composition serves as a convenient and readily accessible method for contacting the article with the composition. It has also been found that microporous structures can readily be generated from the articles at room temperatures, further adding to the convenience. Of course, the temperature may be varied as desired or practicable to achieve a suitable microporous network.

The pore size of the resulting microporous structure may vary depending on, among other things, the composition of the polymeric material, the birefringence of the material, the solvent and non-solvent used, and the like. It has been found that the average size of the pores generated can be moderately controlled by the solvent composition employed. Average pore sizes generated using the methods described herein, in some embodiments, can range from between 1 micrometer to 500 micrometers, such as between 10 and 200 micrometers. While the mechanism of pore formation is not entirely understood, using an alcohol (e.g. isopropanol or ethanol) as a nonsolvent tends to favor the formation of smaller average pore sizes, and water as a nonsolvent tends to favor formation of larger pore sizes on polystyrene substrates.

The resulting microporous structure that forms from the polymeric article may be an interconnected open cell structure or a non-interconnected open cell structure. Again, while the mechanism is not entirely understood, we have found that higher degrees of orientation (higher birefringence) tends to favor formation of more highly interconnected porous structures. Microscopic examination of the microporous structure may give an indication as to whether the resulting microporous structure is interconnected or non-interconnected.

The pore forming process may be ended by any suitable mechanism, such as removing the composition comprising the solvent from the article. The composition may be removed in any suitable manner, such as removing the article from the solvent/non-solvent composition source and drying. Drying may be facilitated by increasing temperature, suction, or blowing air or nitrogen, vacuum stripping, or the like. In embodiments, the article is contacted with a non-solvent composition (e.g., having a Hansen RED for the polymer of about 2.2 or higher) that is miscible with the one or more solvents in the solvent composition to extract the solvent from the article.

In various embodiments, additional treatment well known in the area of cell culture is performed. For example, the surface may be plasma treated, coated with a suitable polymer, polypeptide, carbohydrate, or the like. The additional treatment may be applied before or after the mask is removed.

### 3. Summary of Selected Disclosed Aspects

This disclosure in various aspects describes methods and articles.

In a first aspect a cell culture article is described. The cell culture article includes a three dimensional substrate having a surface for culturing cells, the substrate having one or more optically opaque regions and one or more optically transparent regions, wherein the optically opaque regions occupy 75 percent or more of the surface of the substrate, and wherein at least one of the optically transparent regions occupies an area of the surface sufficient for viewing cells via a light microscope.

A second aspect is an article of the first aspect, wherein the optically opaque regions occupy 95 percent or more of the surface of the substrate.

A third aspect is an article of the first aspect, wherein the optically opaque regions occupy 99 percent or more of the surface of the substrate.

A fourth aspect is a cell culture article of any of the first three aspects, wherein at least one of the optically transparent regions occupies an area of 0.025 mm² or greater.

A fifth aspect is a cell culture article of any of the first four aspects, wherein at least one of the optically transparent regions has a length and width of 0.3 millimeters or greater.

A sixth aspect is a cell culture article of any of the first five aspects, wherein the one or more optically transparent regions form one or more substantially rectilinear lines extending the length of the substrate surface, wherein the lines have a width of 0.2 mm or greater.

A seventh aspect is an article of the sixth aspect, wherein at least two of the optically transparent lines form a cross shape.

An eighth aspect is a cell culture article of any of the first seven aspects, wherein the one or more optically opaque regions provide a three-dimensional microenvironment for cell culture.

An ninth aspect is a cell culture article of any of the first eight aspects, wherein the one or more optically opaque regions are microporous.

A tenth aspect is a cell culture article of any of the first nine aspects, wherein the substrate comprises a polystyrene or a cyclic olefin copolymer.

An eleventh aspect is an article of any of the first ten aspects, wherein the optically opaque regions and the optically transparent regions are formed from the same material or essentially the same material.

A twelfth aspect is an article of any of the first eleven aspects, wherein at least a portion of the surface is plasma treated.

A thirteenth aspect is a method for fabricating a cell culture substrate having one or more optically opaque regions and one or more optically transparent regions. The method includes (i) applying mask to an optically transparent substrate to produce a masked substrate having a masked and an unmasked portion; (ii) applying a treatment to the masked substrate to render the unmasked portion optically opaque; and (iii) removing the mask.

A fourteenth aspect is a method of the thirteenth aspect, wherein applying the treatment consists essentially of contacting the masked substrate with a composition comprising a solvent and removing the composition from the substrate, wherein the substrate is formed from a thermoplastic polymer.

A fifteenth aspect is a method of the fourteenth aspect, wherein the substrate is formed from polystyrene and has a birefringence of 0.0001 or greater.

A sixteenth aspect is a method of the fifteenth aspect, wherein the composition comprising the solvent further comprises a non-solvent.

A seventeenth aspect is a method the sixteenth aspect, wherein the solvent is selected from the group consisting of tetrahydrofuran and ethyl acetate, wherein the non-solvent is selected from the group consisting of water and a C1-C4 unsubstituted alcohol, and wherein the solvent/non-solvent ratio is 30/70 to 70/30 on a volume/volume basis.

An eighteenth aspect is a method of the fourteenth aspect, wherein the substrate is formed from a cyclic olefin copolymer and has a birefringence of 0.0001 or greater.

A nineteenth aspect is a method of the eighteenth aspect, wherein the solvent is selected from the group consisting of methylene chloride and tetrahydrofuran.

A twentieth aspect is a method of the nineteenth aspect, wherein the composition further comprises a non-solvent selected from the group consisting of water and a C1-C4 alcohol.

A twenty-first aspect is a method of any of aspects 13-20, further comprising plasma treating at least a portion of the substrate.

In the following, non-limiting examples are presented, which describe various embodiments of the articles and methods discussed above.

### EXAMPLES

To test the concept of using a region of an optically transparent surface in 3D cell culture substrate for microscopic observation and imaging of cells, a hybrid of 2D and 3D substrate was created by masking a small portion of 2D surface in 6-well polystyrene microplate (Corning Inc). The well bottom was then solvent treated to make the 3D microporous structure with solvent. Preosteoblastic cell line MC3T3 (ATCC) was used to test the hybrid of 2D and 3D substrate.

### Example 1

### Fabrication of 2D and 3D Hybrid Substrate

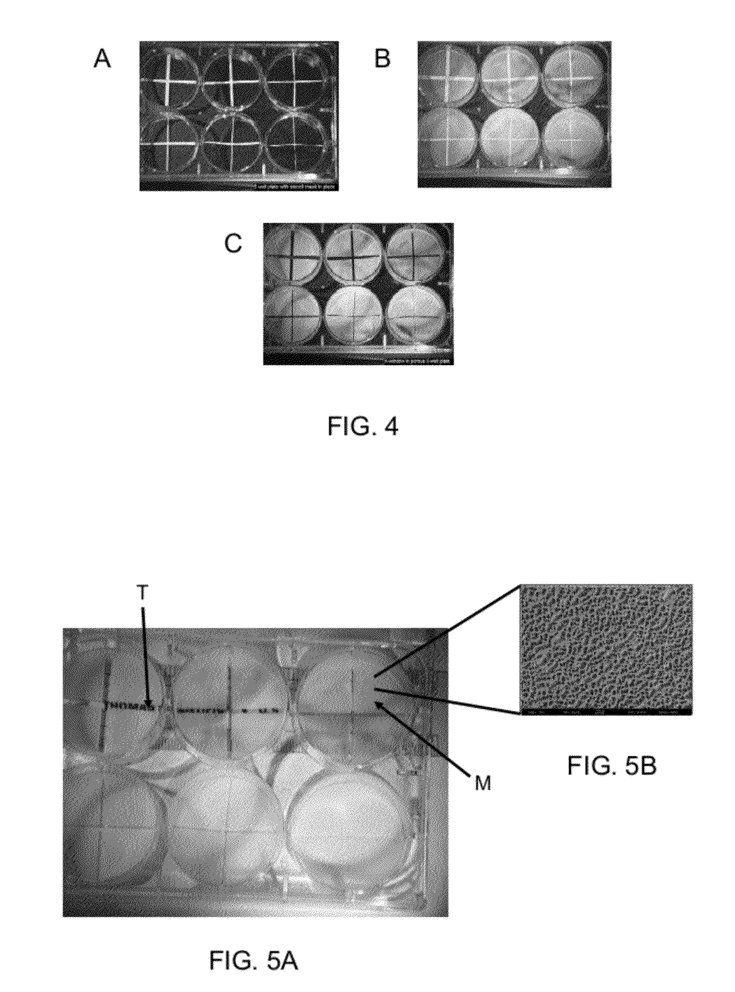

A cross-shaped vinyl adhesive (The Paper Studio, Oklahoma City, Okla., USA) stencil mask was made using a desktop digital craft cutter to a length of 34.8 mm with different widths of 2.0 mm, 1.5 mm, 1.0 mm, 0.8 mm, 0.5 mm and 0.3 mm. We chose different widths to determine whether a cut-off existed regarding a minimum window width necessary to view the cultured cells. A cross-shaped vinyl adhesive was applied firmly to the bottom of each well of a 6-well polystyrene plate (see FIG. 4A), with each well having a different width stencil. Of course, during commercial manufacture and production, the widths of the stencils used in each well may be the same. A cross-pattern was chosen so that the observation window would span an area over the entire length of the well bottom from edge to edge to see the morphology of the cells not only in the middle of the well but also near the wall of the well.

A method as described in co-pending U.S. patent application Ser. No. 13/218,002, entitled MICROPOROUS THERMOPLASTIC ARTICLE, filed on the same date as the present application, naming Michael E. DeRosa and Mircea Despa as inventors, and having attorney docket number SP10-243, which application is hereby incorporated by reference in its entirety to the extent that it does not conflict with the present disclosure, was employed to render microporous the surface of the polystyrene. Briefly, 1 ml of a 40/60 v/v mixture of THF/Isopropanol (Sigma) was pipetted into each well and allowed to sit for 30 seconds. The residual solvent was extracted from each well and the plate was blow dried under nitrogen for approximately 1-2 min (see resulting plates in FIG. 4B). The adhesive stencil mask was then removed to expose the cross shaped 2D polystyrene surface (see FIG. 4C). The plate was then placed in a vacuum oven at 50° C. overnight at 25 inches Hg to remove any residual solvent. The plate was then placed in an RF plasma chamber and oxygen plasma treated for 60 s at 40 W to make the surface hydrophilic before cell culture testing.

Another 6-well polystyrene microplate was solvent treated without the stencil mask with the same mixture of solvents and used as a 3D substrate control.

In this demonstration we used only one mixture of solvent and non-solvent. However, other mixtures of solvent and non-solvent could be used. We found that a mixture of THF and IPA or ethanol worked well. We also found that ethyl acetate as the solvent and IPA or ethanol as the non-solvent worked well too. The ratio of THF/IPA on a vol/vol % basis that works best is 30/70-45/55. The same ratio works for THF/ethanol. We were unable to generate microporous structures on the surface using ratios outside this range. The vol/vol ratios of ethyl acetate/IPA that work best are 45/55-65/35. Again, we were unable to generate microporous structures on the surface using ratios outside this range.

Optical stereo microscopy analysis showed that the microporous structure in the 2D/3D hybrid plate was similar to a plate completely covered by a 3D texture. The 3D polystyrene substrate is not optically transparent, while the 2D polystyrene cross area remains transparent. See FIG. 5A, where the arrow labeled T indicates the transparent area. Label M in FIG. 5A indicates a microporous quadrant. FIG. 5B is a scanning electron micrograph of the indicated portion of the microporous quadrant FIG. 5A

### Example 2

### Cell Culture Testing on the Hybrid 2D/3D Substrate

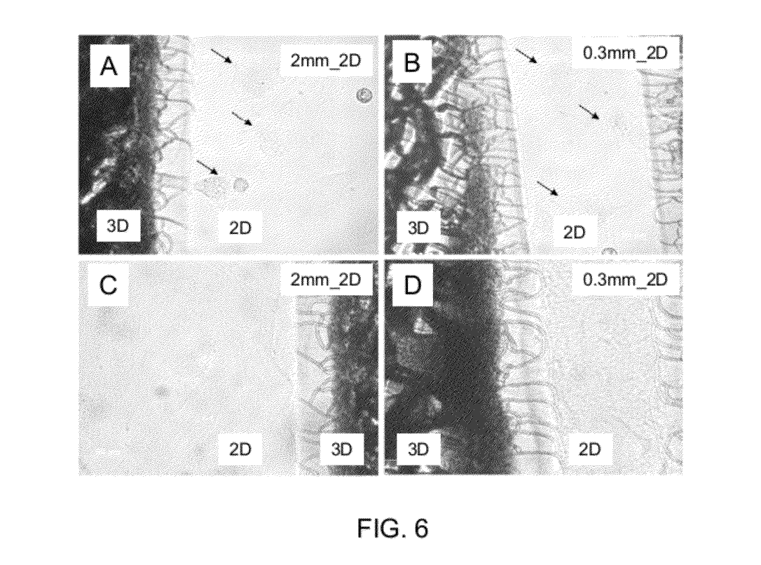

MC3T3 cells were used for testing the 2D/3D hybrid polystyrene substrate. MC3T3 cells were maintained in complete growth medium, ascorbic acid-free alpha Minimum Essential Media (αMEM) supplemented with 10% fetal bovine serum (FBS) and 1% penicillin and streptomycin (Penn/Strep), incubated at 37° C. and 5% CO₂. 1.2×10⁵ cells per well were seeded in 6-well plates. After two hours in growth medium, the pictures of cells were taken using an inverted light microscope (Zeiss Axiovert 200M) with 5×, 10×20× and 40× objectives. All of the widths of the observation windows of transparent 2D surfaces (from 2 mm to 0.3 mm) were large enough to provide sufficient area for microscopic observation and image acquisition at low (5×) or high (40×) magnification. The 3D porous polystyrene was not transparent, making it difficult, it not impossible, to observe the cell behavior and obtain images of cells on opaque 3D polystyrene under the light microscope. The cell morphology and attachment rate on 2D surface with different widths (0.3 mm-2 mm) were similar to TCT and plasma treated 2D (Model MPS-300; March Instruments, Inc., Concord, Calif., USA) polystyrene after 2 hours seeding the cells in growth medium (FIGS. 6A-B). The routine daily cell culture examination was performed by using a phase microscope (Zeiss ID03) with 5×, 10× and 20× objectives through the window of 2D surface.

After two days cells culture, similar cell confluence and morphology were observed and captured using the Zeiss AxioVert 200M microscope (FIGS. 6C-D). Therefore, the transparent 2D surface in opaque 3D polystyrene substrate provides an observation window for examining and imaging the cells in 3D cell cultures. No visible contamination was observed during the cell culture.

### Example 3

### Actin and Nucleus Staining

After 5 days cell culture, MC3T3 cells were washed twice with prewarmed phosphate-buffered saline (PBS), pH 7.4, and then the samples were fixed in 3.7% formaldehyde solution in PBS for 10 minutes at room temperature (RT). Prior to peamealizing the cells with a solution of 0.1% Triton X-100 in PBS for 5 minutes at RT, the cells were rinsed two or more times with PBS. For actin staining, the stock solution of fluorescent FITC labeled phalloidin (Invitrogen) was diluted into 1 to 50 dilutions in PBS. To reduce nonspecific background staining with these conjugates, 1% bovine serum albumin (BSA) was added to the staining solution. 500 μl FITC-phalloidin dilutions were placed into each well of 6-well plate and incubated for 20 minutes at room temperature. To avoid evaporation, cover the plate were covered during the staining and shielded from light using foil paper.

For nuclei staining, the cells were then washed two times with PBS at room before adding 4′-6-Diamidino-2-phenylindole (DAPI) staining solution. 500 ml of DAPI (Vector Laboratories) dilution at 1:5 in PBS was added to each well prior to florescent microscopic observation and imaging.

The 2D window can serve as an observation window for fluorescence (data not shown). We were unable to visualize cells on the 3D surface even after fluorescent staining (data not shown).

### Example 4

### Lactate Dehydrogenase (LDH) Assay

CytoTox 96® Non-Radioactive Cytotoxicity Assay Kit (Promega) was scaled up to test the cell culture in 6-well plates. The cytosolic enzyme LDH is released from the cells using triton-x-100. The colorimetric measurement provides a non-radioactive method for measuring this LDH activity.

Briefly, after 5 days incubation at 37° C. and 5% CO₂, MC3T3 cells were washed with PBS (3 ml/well) and then 500 μl PBS was added to each well alone with 10 μl of 10% of Triton-X-100 (Sigma). 50 μl of cell lysate from each well was transferred to a fresh flat bottom 96-well assay plate (Corning) and then mixed with 50 μl substrate in assay buffer. The reaction was protected from strong direct light by covering the plate with foil paper for 10 minutes at room temperature. 50 μl of stop solution was added to each well and the assay plate was read the absorbance at 490 nm with Wallec plate reader (Perkin Elmer).

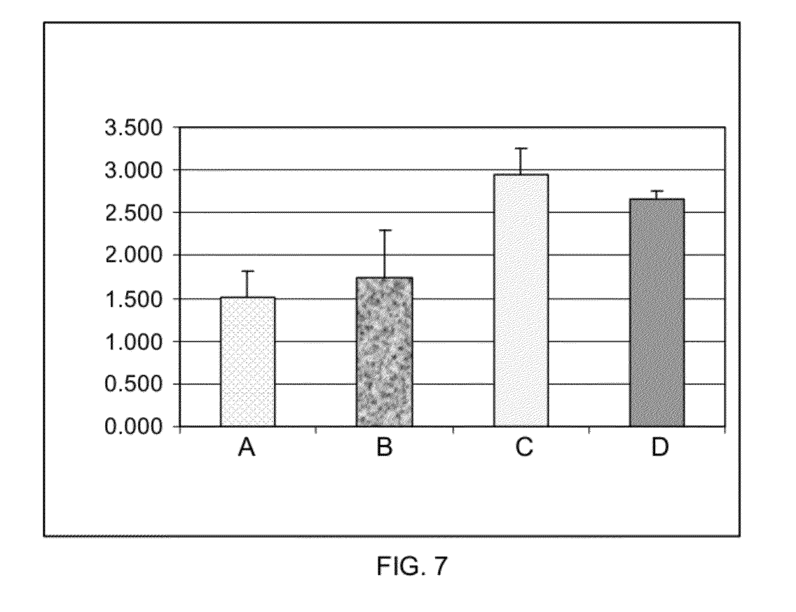

The LDH assay showed the cell number on the 2D3D hybrid substrate was similar to the 3D substrate. The less than 1% 2D surface area inserted into 3D substrate did not significantly affect the 3D cell culture (FIG. 7). In FIG. 7, the Y-axis is the optical density at 490 nanometers; (A) is the hybrid 2D/3D substrate; (B) is the 3D substrate; (C) is a plasma treated polystyrene substrate (Model MPS-300; March Instruments, Inc., Concord, Calif., USA); and (D) is a TCT-treated polystyrene substrate (Corning Incorporated).

### Example 5

### Hansen Solubility Parameters for Solvents that Form Microporous Surfaces on Polystyrene Articles

As described in more detail in co-pending U.S. patent application Ser. No. 13/217,818, entitled MICROPOROUS THERMOPLASTIC SHEETS, having attorney docket no. SP11-197 naming Michael DeRosa, Todd Upton, and Ying Zhang as inventors, and filed on the same date herewith, we performed testing to determine which solvents or mixtures of solvents and non-solvents formed microporous surfaces on polystyrene articles and determined the Hansen RED values of those solvents and solvent/non-solvent mixtures that were effective in pore formation. A brief overview of those studies is presented herein.

Briefly, Hansen solubility parameters for solvent mixtures that form microporous surfaces on a molded polystyrene cell culture plate, which had a gradient of birefringence values across the surface (with a significant portion being greater than 0.001), were determined in the following manner. First, a range of known solvents and non-solvents for polystyrene were tested on the surface of a molded polystyrene cell culture plate. 50-100 microliters of each test solvent and non-solvent were pipetted onto the surface of the polystyrene at room temperature. Observations were made under a microscope to see if the solvent dissolved the surface within a 2 min time period. Once a range of solvents and non-solvents were tested (see Table 1), the Hansen parameters, δP and δH, were plotted against each other for each test solvent. This type of two dimensional plot shows one cross section of the total three dimensional polystyrene solubility sphere.

TABLE 1
Solvents and non-solvents used to determine Hansen Solubility Parameters

Solvents
1,1,1-Trichloroethane
Methylene Dichloride (Dichloromethane)
N-Methyl-2-Pyrrolidone
Ethyl Acetate
Dimethylformamide
n-Butyl Acetate
Chlorobenzene
Cyclohexanone
Isoamyl Acetate
1,3-Dioxolane
Toluene
Acetone
1,1-Dichloroethane
Tetrahydrofuran
Diethyl Ether
Methyl Ethyl Ketone
Non solvents
Cyclohexane
2-Propanol
Ethyl Lactate
Methanol
Dimethyl Sulfoxide
Glycerol
Water
Propylene Carbonate
1-Butanol
Ethanol

Using HSPiP software (Hansen Solubility Parameters in Practice, v.3.1) a fit of the data was calculated to determine the center coordinates of the polystyrene sphere and the solubility radius Rₒ. Data analysis using HSPiP software found the parameters to be δ_{D}=16.98, δ_{P}=6.76 and δ_{H}=4.83 with Rₒ=6.4. 50-100 microliters of solvent/nonsolvent mixtures including tetrahydro furan/water, tetrahydrofuran/isopropanol, tetrahydrofuran/propylene carbonate, ethylacetate/isopropanol, toluene/dimethyl sulfoxide, acetone/isopropanol, and 1,3 dioxolane/water were pipetted onto the polymer surface allowed to sit for 60 seconds then blow dried. The resulting surface features were observed under a microscope.

HSPiP software was used to determine the Hansen solubility parameters of the solvent/nonsolvent mixtures with the v/v % ranges shown in Table 2. The solubility parameters of the mixtures were plotted against the known solvent and non-solvent values determined earlier. A solubility boundary having a radius Rₒ=6.4 was determined. It was also found that the solubility parameter range of the solvent/non-solvent mixtures that formed microporous surfaces have RED values in the range of 0.88-1.41.

TABLE 2
Solvents with appropriate RED values to form microporous polystyrene

| Solvent/Non-solvent mixture | Range v/v % |
|---|---|
| Tetrahydrofuran/water | 50/50-65/35 |
| Tetrahydrofuran/isopropanol | 35/65-45/55 |
| Tetrahydrofuran/propylene carbonate | 37/63-50/50 |
| Ethylacetate/isopropanol | 60/40-70/30 |
| Toluene/dimethyl sulfoxide | 25/75-30/70 |
| Acetone/isopropanol | 70/30-80/20 |
| 1,3 Dioxolane/water | 60/40-80/20 |

While the polymeric articles tested in this example were molded cell culture articles, the Hansen solubility parameters should be representative of other polymeric articles.

Thus, embodiments of POROUS SUBSTRATE FOR CELL CULTURE WITH NON-POROUS REGION are disclosed. One skilled in the art will appreciate that the cell culture apparatuses and methods described herein can be practiced with embodiments other than those disclosed. The disclosed embodiments are presented for purposes of illustration and not limitation.

What is claimed is:

1. A cell culture article comprising:
a three dimensional substrate having a surface for culturing cells, the substrate having one or more optically opaque regions and one or more optically transparent regions, wherein the optically opaque regions occupy 75 percent or more of the surface of the substrate, and wherein at least one of the optically transparent regions occupies an area of the surface sufficient for viewing cells via a light microscope.

2. A cell culture article according to claim 1, wherein the optically opaque regions occupy 95 percent or more of the surface of the substrate.

3. A cell culture article according to claim 1, wherein the optically opaque regions occupy 99 percent or more of the surface of the substrate.

4. A cell culture article according to claim 1, wherein at least one of the optically transparent regions occupies an area of 0.025 mm² or greater.

5. A cell culture article according to claim 1, wherein at least one of the optically transparent regions has a length and width of 0.3 millimeters or greater.

6. A cell culture article according to claim 1, wherein the one or more optically transparent regions form one or more substantially rectilinear lines extending the length of the substrate surface, wherein the lines have a width of 0.2 mm or greater.

7. A cell culture article according to claim 6, wherein at least two of the optically transparent lines form a cross shape.

8. A cell culture article according to claim 1, wherein the one or more optically opaque regions provide a three-dimensional microenvironment for cell culture.

9. A cell culture article according to claim 1, wherein the one or more optically opaque regions are microporous.

10. A cell culture article according to claim 1, wherein the substrate comprises a polystyrene or a cyclic olefin copolymer.

11. A cell culture article according to claim 1, wherein the optically opaque regions and the optically transparent regions are formed from the same material or essentially the same material.

12. A method for fabricating a cell culture substrate having one or more optically opaque regions and one or more optically transparent regions, comprising:
applying mask to an optically transparent substrate to produce a masked substrate having a masked and an unmasked portion;
applying a treatment to the masked substrate to render the unmasked portion optically opaque; and
removing the mask.

13. The method of claim 12, wherein applying the treatment consists essentially of contacting the masked substrate with a composition comprising a solvent and removing the composition from the substrate, wherein the substrate is formed from a thermoplastic polymer.

14. The method of claim 13, wherein the substrate is formed from polystyrene and has a birefringence of 0.0001 or greater.

15. The method of claim 14, wherein the composition comprising the solvent further comprises a non-solvent.

16. The method of claim 15, wherein the solvent is selected from the group consisting of tetrahydrofuran and ethyl acetate, wherein the non-solvent is selected from the group consisting of water and a C1-C4 unsubstituted alcohol, and wherein the solvent/non-solvent ratio is 30/70 to 70/30 on a volume/volume basis.

17. The method of claim 12 wherein the substrate is formed from a cyclic olefin copolymer and has a birefringence of 0.0001 or greater.

18. The method of claim 17, wherein the solvent is selected from the group consisting of methylene chloride and tetrahydrofuran.

19. The method of claim 18, wherein the composition further comprises a non-solvent selected from the group consisting of water and a C1-C4 alcohol.

20. The method of claim 12, further comprising plasma treating at least a portion of the substrate.
